(19) United States
(12) Patent Application Publication
(10) Pub. No.: US 2012/0150132 A1
(43) Pub. Date: Jun. 14, 2012

(54) DISPOSABLE DISPENSER DROP DIVERTER

(76) Inventor: Jonathan H. Cress, Santa Cruz, CA (US)

(21) Appl. No.: 13/324,801

(22) Filed: Dec. 13, 2011

Related U.S. Application Data
(60) Provisional application No. 61/422,596, filed on Dec. 13, 2010.

(51) Int. Cl. A61F 9/00; A61M 35/00

(52) U.S. Cl. 604/290; 604/298

(57) ABSTRACT

An attachment for use with a unit dose eye drop dispenser comprises a tapered receptacle and a lateral positioning arm. The tapered receptacle is “squeezable” so that a reservoir of the unit dose dispenser can be placed within the receptacle and the walls of the receptacle squeezed to dispense a droplet. The positioning arm is shaped to be placed over the patient's nosebridge to properly position the receptacle over the eye for optimum placement of an eye drop.

### CROSS-REFERENCE TO RELATED APPLICATION

This application claims the benefit of U.S. Provisional Application No. 61/422,596 (Attorney Docket No. 33349-707.101), filed Dec. 13, 2010, the full disclosure of which is incorporated herein by reference.

### BACKGROUND OF THE INVENTION

1. Field of the Invention

The present invention relates generally to medical apparatus and methods. More particularly, the present invention relates to eyedroppers and more specifically to attachments used with unit dose dispensers to deliver drops to the inner corner of a patient's eye.

Many if not most people find dispensing eye drops into their own eyes to be an uncomfortable procedure. Many people blink or shake as the drops are released from the conventional eyedropper, often leading to incomplete delivery of the irrigant, lubricant or medication.

Despite many attempts to provide improved systems, the most common technique for self-dispensation of eye drops remains use of a dropper bottle. The patient turns his or her eyes upwardly, locates a dispensing nozzle of the dropper over the eye, and squeezes the bottle to dispense the drop. Sometimes this works. Sometimes this doesn't work.

Prior attempts at improving eyedroppers and systems are almost too numerous to mention. Of particular interest to the present invention, a variety of eye drop dispensing guides or positioners have been developed. For the most part, these improved guides or dispensers have been attempts to stabilize and/or position the dispensing nozzle of the dropper or dropper bottle at some predetermined position over the eye. In some cases, the guides or positioners have further included means for holding the eyelid open in an attempt to assure that the drop actually enters the eye.

A particular attachment intended for use with conventional multi-dose eyedropper bottles is described in U.S. Pat. No. 7,309,329, invented and owned by the inventor of the present application. U.S. Pat. No. 7,309,329, describes a Y-shaped attachment having a pair of arms which are placed over a patient's nosebridge. A multi-dose dropper bottle is secured to a center fixture and dispenses a drop that travels down a passage formed in one of the legs. The passage releases the drop into a corner of the eye.

While a significant advance over other positioners and eyedropper aids, the structure of the attachment described in U.S. Pat. No. 7,309,329 is not well suited for use with unit dose eye drop dispensers. Such dispensers 10, as illustrated in FIG. 1, comprise a squeezable reservoir 12, a holding tab 14, a break tab 16, and a dispensing tip 20. The reservoir holds a singe dose of fluid to be dispensed, typically from 40 μl to 60 μl, and the user breaks off the tab 16 along break line 18 to open a small dispensing aperture in the dispensing tip 20. By holding the reservoir 12 between the thumb and forefinger, the user can locate the dispenser over the eye and squeeze the reservoir to dispense the single drop. The dispenser can then be disposed. Such unit dose dispensers thus suffer from all of the problems discussed above with respect to multi-dose dispensing bottles, and further suffer from being small and difficult to manipulate. Patients having limited dexterity from arthritis or other causes can have particular difficulties both in placing these devices and in squeezing sufficiently to dispense the entire drop.

For these reasons, it would be desirable to provide additional and improved devices and apparatus for accurately and successfully dispensing drops into a patient's eyes. In particular, it would be desirable if the apparatus and systems were useful with unit dose dispensers, could provide accurate and complete delivery of a drop from the unit dose dispenser to a target area of the eye, particularly to the inner corner (medial canthus) of the eye. At least some of these objectives will be met by the invention as described and claimed hereinbelow.

2. Description of the Background Art

U.S. Pat. No. 7,309,329 has been described above. Additional patents describing eyedroppers and other devices for directing drops into eyes include U.S. Pat. Nos. 6,595,970; 6,325,784; 6,319,240; 5,713,495; 5,665,079; 5,059,188; 4,973,322; 4,960,407; 4,685,906; 4,468,103; 4,257,417; 4,085,750; and 3,934,590. See also published application US 2002/0016576 and design Pat. No. D463,550; D457,952; and D369,211. The full disclosures of each of these patents and applications are incorporated herein by reference.

### BRIEF SUMMARY OF THE INVENTION

The present invention provides a drop dispensing attachment and methods for it's use with a unit dose drop dispenser intended for delivering drops to an eye. Unit dose or “single drop” eye drop dispensers are small, soft plastic units that hold a single dose of a fluid intended to be delivered to an eye, such as a lubricant, medication, or the like. The unit dose dispensers have a tab which includes labeling and which can assist in handling the very small package. After a distal tab is broken off to open the distal tip of the dispenser for use, the user must hold the receptacle over an eye and simultaneously squeeze the walls of the reservoir to dispense the drop.

The present invention provides an attachment which can simplify both the handling and the dispensing of a drop from such unit dose dispensers. In particular, the present invention provides a drop dispensing attachment for use with such unit dose dispensers. The dispensing attachment comprises a tapered receptacle having a squeezable wall, an interior space or volume capable of receiving the reservoir of the unit dose dispenser, and an aperture at one end which allows a drop from the unit dose dispenser to pass out from the receptacle. The drop dispensing attachment further includes a positioning arm projecting laterally from a side of the tapered receptacle so that the aperture of the receptacle is positioned or located over a patient's eye when the arm is resting over the patient's nosebridge.

At least the tapered receptacle of the unit dose dispenser will typically be formed from an elastomer, such as a silicone rubber, latex, or the like. Preferably, the entire unit dose dispenser including the reservoir and the positioning arm will be formed from the elastomer, more typically being molded from the elastomer as a monolithic or integrated structure, i.e., free from seams and discontinuities.

In specific examples of the unit dose dispenser, a lower extension may be formed adjacent to the aperture at the lower end of the tapered receptacle. The lower extension will include a surface which guides the drop after it passes through the aperture. Usually, the surface will be inclined laterally away from the aperture toward the side of the positioning arm. As described in more detail below, such a configuration will help guide the drop toward the inner corner of the eye after it emerges from the aperture in the reservoir. In particular, the drop will adhere to the surface by surface tension to follow the surface to the desired release point.

In a further aspect of the present invention, a method for dispensing a fluid drop into the eye of a patient comprises inserting a unit dose dispenser into an interior of a tapered receptacle. A positioning arm extending laterally from the tapered receptacle is placed over the patient's nosebridge to align a dispensing tip of the unit dose dispenser over the patient's eye. The wall of the receptacle is squeezed to in turn squeeze the reservoir of the unit dose dispenser and release a drop through an aperture in the tapered receptacle and into the patient's eye.

In preferred aspects of the methods of the present invention, the drop falls into an inner corner of the patient's eye. More particularly, the drop may be released onto a lower extension surface of the receptacle to guide the drop into the inner corner of the patient's eye. In this way, the drop can be guided to the innermost corner of the eye to maximize patient comfort.

The drop dispensing attachments and methods of present invention provide a number of advantages. In particular, the positioning arm of the attachment allows the user to easily locate the unit dose dispenser near the corner of the eye to achieve an optimum release point. With the preferred curved or semi-circular positioning arm, the unit dose dispenser can be easily moved over the nosebridge until the lower extension engages the side of the nose for optimum placement. Additionally, the receptacle of the drop dispensing attachment which is placed over the reservoir of the unit dose dispenser increases the size of the assembly, making the assembly easier to manipulate than the unit dose dispenser by itself. By further providing an elastomer having a slightly “tacky” surface, such as silicone rubber, the ease of manipulating the assembly is further enhanced. Additionally, the ability to produce the drop dispensing attachment as a simple, molded silicone rubber or other elastomer part sufficiently reduces its cost so that it can be disposed of together with the unit dose dispenser. Disposability is a particular advantage in order to maintain the sterility of the unit dose dispensers.

### BRIEF DESCRIPTION OF THE DRAWINGS

FIG. 1 (prior art) illustrates a unit dose dispenser of the type useful with the apparatus and methods of the present invention.

FIG. 2 is a side view of a drop dispensing attachment constructed in accordance with the principles of the present invention.

FIG. 3 is a partial cross-sectional view of the drop dispensing attachment of FIG. 2.

FIG. 4 is a cross-sectional view taken along line 4-4 of FIG. 2.

FIG. 5 illustrates an assembly of the unit dose dispenser of FIG. 1 placed in the drop dispensing attachment of FIGS. 2-4.

FIGS. 6A-6D illustrate use of the drop dispensing assembly of FIG. 5 for placement of a single drop in the inner corner of a patient's eye in accordance with the principles of the present invention.

### DETAILED DESCRIPTION OF THE INVENTION

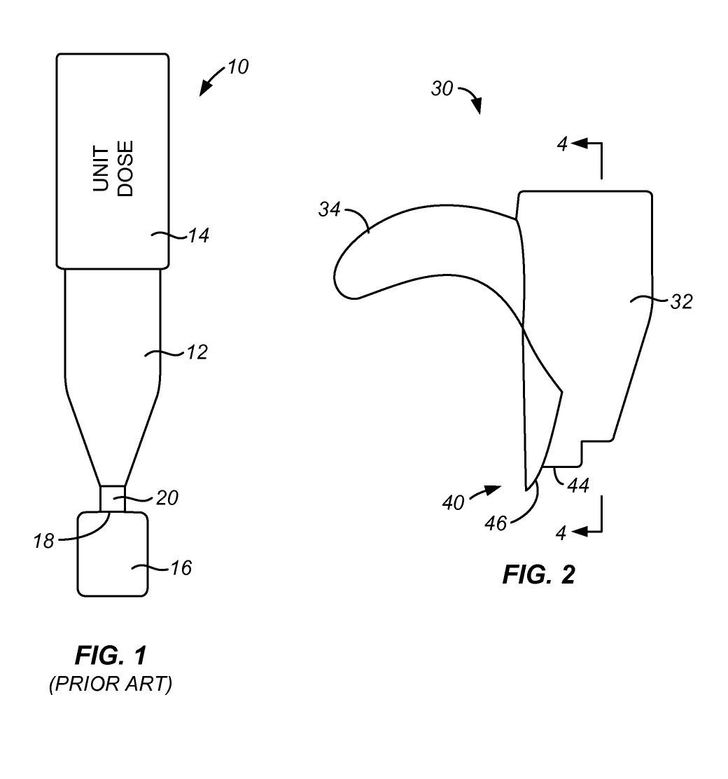

The unit dose dispenser 10 illustrated in FIG. 1 has been described above.

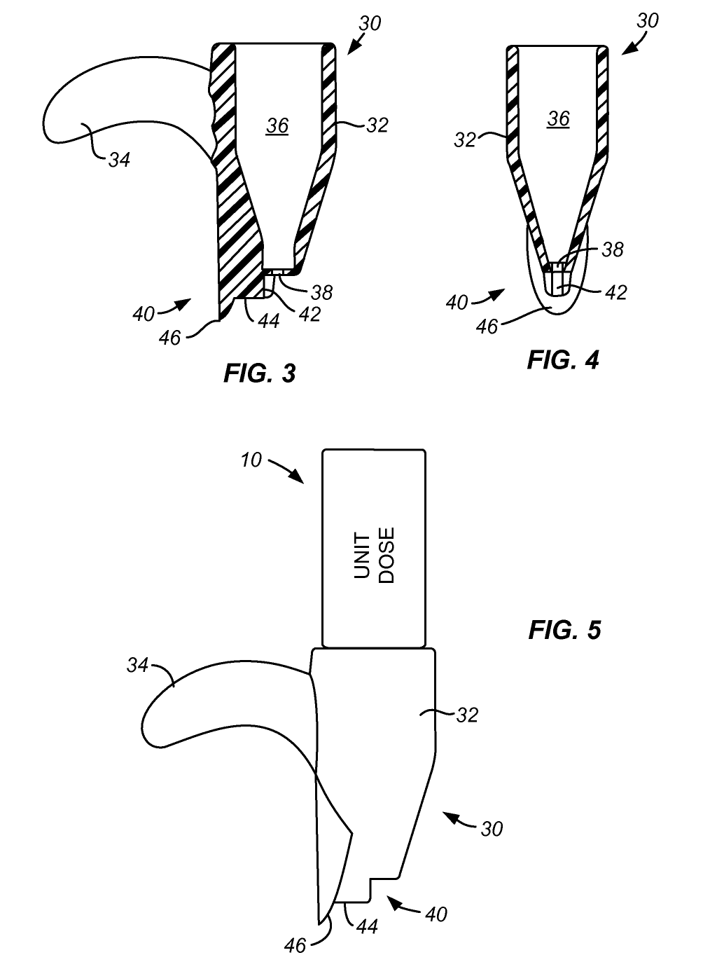

A drop dispensing attachment 30 intended to receive and be used with the unit dose dispenser 10 is illustrated in FIGS. 2-4. The drop dispensing attachment 30 comprises a tapered receptacle 32 and a positioning arm 34 extending laterally from a side of the tapered receptacle. The tapered receptacle 32 defines an inner cavity 36 (FIGS. 3 and 4) having a tapered geometry which generally corresponds to the exterior shape of the reservoir 12 of a conventional unit dose dispenser. It will be appreciated that to the extent such unit dose dispensers have different geometries, or the geometries may change in the future, the shape of the inner cavity 36 can be adjusted accordingly.

The walls of the tapered receptacle 32 will be sufficiently compliant and flexible so that they are “squeezable” when the drop dispensing attachment 30 is placed over the unit dose dispenser, as best seen in FIG. 5. Typically, at least the tapered reservoir 32 of the drop dispensing attachment 30 will be formed from a relatively soft elastomer, such as silicone rubber, latex, or the like, usually having durometer (Shore A hardness) in the range from 10 to 90, more usually from 30 to 60. While the walls of the tapered receptacle 32 could have a more complex geometry, such as having pleated or accordion regions, partially reinforced regions, or the like, it will usually be preferred to have the walls formed from the elastomer, free from reinforcements and features, so that the wall of the tapered receptacle can be slightly stretched over the exterior of the reservoir 12 of the unit dose dispenser 10 as the receptacle is placed therein.

The drop dispensing attachment 30 will further include the laterally placed positioning arm 34. The positioning arm is typically formed integrally or “monolithically” with the body of the tapered receptacle 32, preferably, both the positioning arm and the tapered receptacle will be formed in a single molding step from a single material or blend. The positioning arm will preferably have a slight curve or arc. In the preferred embodiments, the tapered receptacle will have a length in the range from 2.5 cm to 4 cm and the positioning arm will extend laterally from the wall of the tapered receptacle by a distance in the range from 1.5 cm to 2.5 cm. The arc of the inner radius of the positioning arm will typically have a radius in the range from 1 cm to 1.5 cm. While the positioning arm will usually be free from reinforcements, it some cases it may be desirable to provide an inner core or other reinforcing element, but such reinforcement will typically not extend into the wall of the tapered receptacle 32.

An opening or aperture 38 is formed at the lower end of the inner cavity 36 and the tapered receptacle 32 of the drop dispensing attachment 30, as seen in FIGS. 3 and 4. The open end of the distal tip 20 of the unit dose dispenser 10 will lie immediately above the aperture 38 when the unit dose dispenser 10 is present in the drop dispensing attachment as illustrated in FIG. 5.

A lower extension 40 is usually provided at the lower end of the tapered receptacle 32 of the drop dispensing attachment 30, as best seen in FIGS. 3 and 4. The lower attachment 40 defines a channel 42 which allows the drop emerging from aperture 38 to fall vertically downward in use. As the drop reaches the bottom end of the channel 42 it is carried by surface tension along lateral surface 44 of the lower extension 40 until it reaches an inclined surface 46. In all cases, the drop adheres to the surfaces by surface tension.

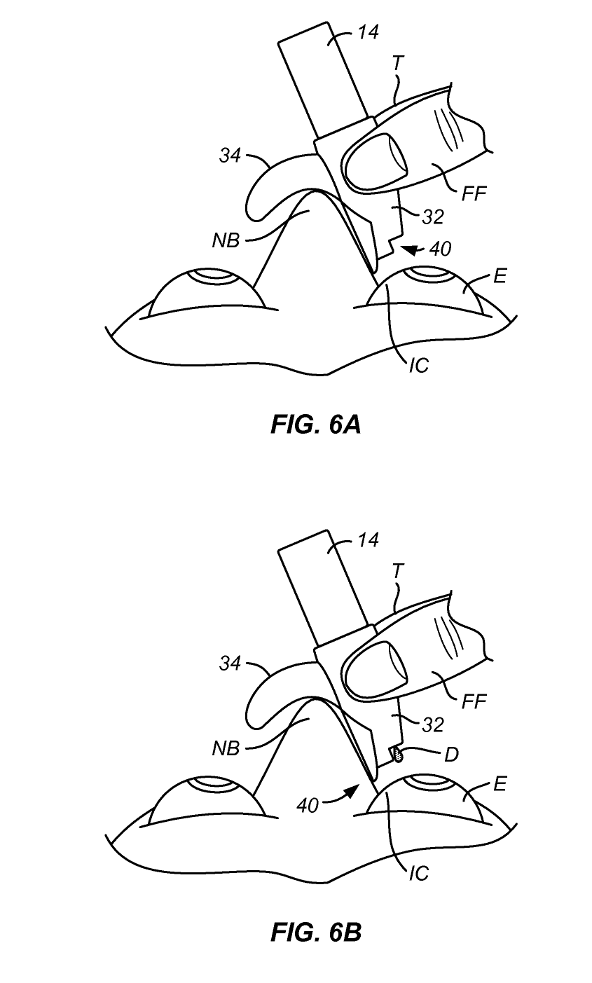

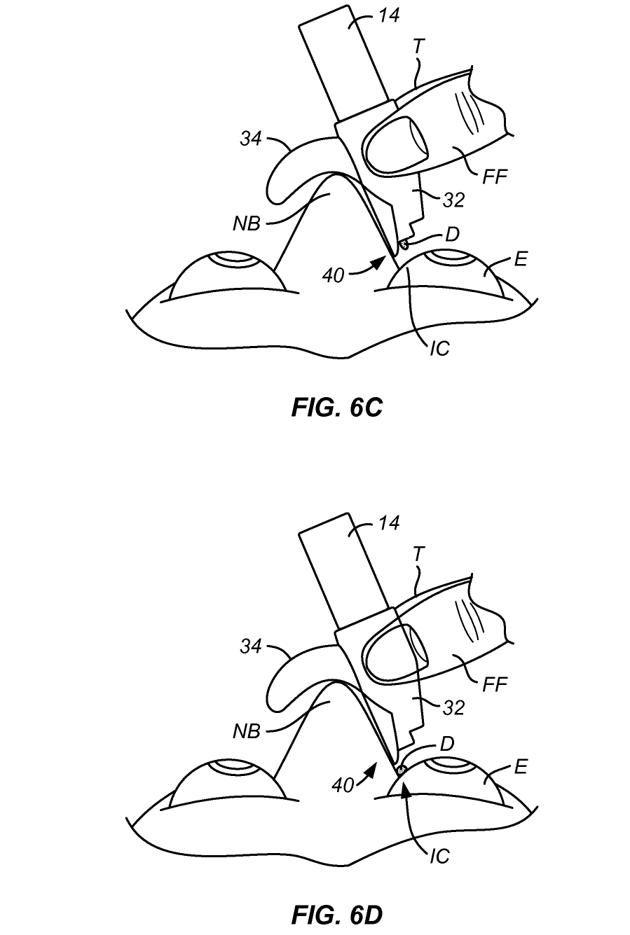

Use of the assembly of the unit dose dispenser 10 and drop dispensing attachment 30, is shown in FIG. 5, for dispensing a drop D into a patient's eye E is illustrated in FIGS. 6A-6D. After breaking off the tab 16 from the unit dose dispenser 10 and inserting the dispenser into the drop dispensing attachment 30, the user holds the tapered receptacle 32 of the assembly between the user's forefinger FF and thumb T, as shown in FIG. 6A. A lower curved surface of the positioning arm 34 is placed over the nosebridge, and the assembly is positioned so that the lower extension 40 engages a side of the nose positioning the extension over the inner corner IC of the patient's eye E, also as shown in FIG. 6A. Once in the position as shown in FIG. 6A, the user is ready to dispense the single drop, as illustrated in FIG. 6B-6C. To dispense the drop, the user squeezes the “squeezable” walls of the tapered receptacle 32 using the forefinger FF and thumb T to cause the walls of the reservoir of the unit dose dispenser to collapse and release a drop D through the aperture 38 of the receptacle (FIGS. 3 and 4) so that it emerges in the channel 42 (FIGS. 3 and 4), as illustrated in FIG. 6B. The drop D then flows from the channel 42 and adheres to the lateral surface 44, as shown in FIG. 6C and then down the inclined surface 46 and into the inner corner IC of the eye E, as shown in FIG. 6D. Dispensing of the single dose drop D is thus greatly simplified and optimally positioned.

What is claimed is:

1. A drop dispensing attachment for a unit dose dispenser, said attachment comprising:
a tapered receptacle having a squeezable wall, an interior capable of receiving a unit dose dispenser, an aperture at one end which allows a drop to pass; and
a positioning arm projecting laterally from the tapered receptacle so that aperture of the receptacle is positioned over a patient's eye when the arm is rested over the patient's nosebridge.

2. A top drop dispensing attachment as in claim 1, wherein at least the tapered receptacle is formed from an elastomer.

3. A drop dispensing attachment as in claim 2, wherein the tapered receptacle and the positioning arm are formed from the elastomer.

4. A drop dispensing attachment as in claim 3, wherein the tapered receptacle and positioning arm are molded from the elastomer.

5. A drop dispensing attachment as in claim 4, wherein the elastomer is selected from the group consisting of a silicone rubber, and latex.

6. A drop dispensing apparatus as in claim 5, wherein the surface is inclined laterally away from the aperture toward the side having the positioning arm to guide the drop to the innermost end of the inner corner of the eye.

7. A method for dispensing a fluid drop into the eye of a patient, said method comprising:
inserting a unit dose dispenser into an interior of a tapered receptacle;
placing a positioning arm extending laterally from the tapered receptacle over the patient's nosebridge to align a dispensing tip of the unit dose dispensing tip of the unit dose dispenser over the patient's eye;
squeezing the wall of the receptacle to in turn squeeze the unit dose dispenser and release a drop through an aperture in the tapered receptacle and into the patient's eye.

8. A method as in claim 7, wherein the drop falls into an inner corner of the patient's eye.

9. A method as in claim 8, wherein the drop released onto a lower extension surface of the receptacle to guide the drop into the inner corner of the patient's eye.
